(19) United States
(12) Patent Application Publication
(10) Pub. No.: US 2014/0303043 A1
(43) Pub. Date: Oct. 9, 2014

(54) REVERSIBLE REACTION SENSORS AND ASSEMBLIES

(71) Applicant: Clear Align LLC, Eagleville, PA (US)

(72) Inventor: Dean Paul Kossives, Glen Gardner, NJ (US)

(21) Appl. No.: 14/315,458

(22) Filed: Jun. 26, 2014

Related U.S. Application Data
(62) Division of application No. 12/807,340, filed on Sep. 2, 2010, now abandoned.
(60) Provisional application No. 61/275,867, filed on Sep. 2, 2009.

(51) Int. Cl. G01N 21/78; G01N 33/00

(52) U.S. Cl. CPC G01N 21/783; G01N 33/0057
USPC 506/39; 422/69

(57) ABSTRACT

A reversible reaction sensor provides for detecting medical, biologic or explosive airborne compounds. The sensor may be formed in semiconductor material and is activated by radiation from sources to provide sensing of particular airborne compounds optically detectable by detectors, and reversed by other radiation from a source (or removal of activating radiation) to take away such airborne compounds from the sensor. The reversible reaction sensor device has a sensing material of one or more photo-chromic or photo-biologic compounds for a specific compound(s) or analytic(s) having receptor sites which bound molecularly to specific compound(s) or analytic(s) when present, in which sensing relies only on the response time to saturation for the sensing material as measured by an optical property of the sensing material when exposed to radiation. The sensing material is self cleaning by one or more of light of a specific wavelength, radio waves of a specific frequency, absence of said light illuminating said material, absence or presence of a magnetic field, which causes the receptor sites to close and the bound specific analytic are released and swept away by flow of air. The sensor may be in hermetically sealed and non-hermetic assemblies.

This Application claims priority to U.S. Provisional Application No. 61/275,867, filed Sep. 2, 2009, which is herein incorporated by reference.

### FIELD OF THE INVENTION

The present invention relates to reversible reaction sensors which are utilized to create to provide medical, biological, material, explosive, and air borne compound sensing. Additionally assemblies of these sensor is provided which allows for specific functional blocks to be manufactured separately and brought together at the final package to form a specific sensor. The assemblies provide packaging which facilitates high volume manufacturing and low cost. Further, hermetic and non-hermetic sensor unit assemblies are provided, which may differ by surface reaction or flow through reaction.

### BACKGROUND OF THE INVENTION

In recent years the need for optical sensor systems has grown due to the increased threat from worldwide terroristic activities. Airport security checkpoints rely on x-ray and Gas Chromatography/Mass Spectrometry (GC/MS) machines to detect and measure the levels (if any) of explosives residues on luggage and other suspect articles. While these methods are accurate and relatively fast, the equipment that accomplishes these tasks is expensive, bulky, and draws large amounts of electrical current. Attempts to produce portable, hand-held versions of these instruments, while successful, are still expensive, and require recharge after only a few hours use. Moreover, these units have limited application in covert surveillance environments due to physical size, and even less utility in battlefield applications due, in large part, to detector swamping by the large quantities of explosives residues found in these environments. To service these needs and enable affordable detection systems for use in military, homeland security, and other markets a fundamental change is desired in the approach to sensors and systems which enables low cost, high volume production. Most importantly, a detection methodology scheme is desired which is not constrained by the need for mass spectrometers, infrared analyzers, large computers, or any combination of the aforementioned technological building blocks.

While many inroads have been made in the integration of optical sensors and electronics, practical, low cost sensors for medical, materials, and biologic detection have yet to be realized and mass produced due to reliance on older detection methodologies such as mass spectrometry and gas chromatography.

### SUMMARY OF THE INVENTION

Accordingly, it is feature of the present invention to provide a sensor whose functionality is not reliant on mass spectrometric or ionization methods.

It is another feature of the present invention to provide a sensor which can be combined with a substrate technology which allows for separately optimized control circuits and standardized advanced sensors to be brought together in sensor assemblies or packaging providing modular optical sensors.

Briefly described, a reversible reaction sensor is provided for detecting medical, biologic or explosive airborne compounds. The reversible sensor may be formed in semiconductor material and is activated by radiation from sources to provide sensing of particular airborne compounds optically detectable by detectors, and reversed by other radiation from a source (or removal of activating radiation) to take away such airborne compounds from the sensor. The reversible reaction sensor device has a sensing material, such as a polymer, of one or more photo-chromic or photo-biologic compounds for a specific analytic having receptor sites which bound molecularly to said specific analytic when present, in which sensing relies only on the response time to saturation for the sensing material as measured by an optical property of the sensing material when exposed to light, and the time to saturation is proportional to the surface area and concentration of the sensing material. The sensing material is self cleaning by one or more of light of a specific wavelength, radio waves of a specific frequency, absence of said light illuminating said material, absence or presence of a magnetic field, which causes the receptor sites to close and the bound specific analytic are released and swept away by flow of air. Hence, the sensor is a reversible reaction sensor.

The sensor unit assembly of the present invention has a sensor element carrier (or member) with channels or V cross-sectional shaped grooves metalized to reflect light down the grooves to provide a waveguide. In each of the one or more grooves is a sensor element (e.g., a glass bead or fiber, or the groove's reflective surface itself) having a photochromic or photobiologic material as a coating or layer. The groove has one end having an angled surface to direct radiation (e.g., light) received from a radiation source toward the other end of the groove. The photochromic or photobiologic material of the sensing element is responsive to the radiation causing particular airborne compound(s) or analytic(s) when present to chemically bond onto the sensor element and cause a change in an optical characteristic (i.e., absorption spectrum, refractive index, or color) of the photochromic or photobiologic material of the sensing element. These particular compound(s) or analytic(s) are preselected in accordance with the photochromic or photobiologic material of the sensing element. The other end of the groove has an angled surface to direct radiation from the sensor element to a detector for use in measuring the presence or level of those particular compound(s) or analytic(s). By removal or changing the radiation to the sensor element releases the airborne compound(s) which bonded to the sensor element. The radiation source and detector for each sensor element may be mounted upon a substrate with pathways (openings or holes) for radiation to pass there though. The substrate is spaced a small distance from the sensor element and its carrier to provide a region for air flow therebetween. Airflow may be provided by a micro fan at one end of the region and an intake filter at the other end of the region.

Another sensor unit assembly of the present invention is also provided having a hermetically sealed housing with a window upon which the photochromic or photobiologic material described above is provided. The same radiation source and detector as described earlier are provided in the housing. Unlike the earlier described non-hermetically sealed sensor unit assembly, no air flow is provided in the housing. In this hermetically sealed sensor unit assembly, a substrate is provided with a channel or V cross-sectional shaped groove providing a waveguide, and sources providing light to the waveguide. Along each groove is an optic element (or director, mirror, or reflective surface) which directs light received along the groove from a light source at one end thereof to the optical window and another optical element (or director, mirror or reflective surface) which receive light from the window and passes the light along the groove to a detector at the other end of the groove.

This hermetically sealed sensor unit assembly may have optical paths from light sources and detectors for non-interferometric detection or interferometric detection of optical changes in the photochromic or photobiologic material. For interferometric detection, radiation is combined from two sources, and then optically split the light into two branches. In one branch the optical elements (or directors) direct and receive light from the window, while the other branch travels along a waveguide having metals acting as a resistive heater or shifter of magnetic field to cause a phase shift in light along the waveguide. The ends of the two branches optically combine to generate interferometric patterns detectable by detectors. Another waveguide may also be provided where reference and object beams are split and then combined between sources and detectors.

In both the hermetically sealed and non-hermetically sealed sensor unit assemblies described above, when the radiation sources are light sources, the sources may be LEDs, Super LED, or other sources that apply radiation at a proper wavelength that enables the surface of the sensing element to respond when airborne compound(s) bond on the surface in accordance with the particular photochromic or photobiologic reaction desired. The light source or other sources may also be provided on substrates to apply radiation (or presence or absence of a magnetic field) which restore the surface of the sensing element, such as to reset the sensor element.

The photochromic or photobiologic material may be a single coating, layer, or multiple materials in layers which are built up on surface of the sensing element, e.g., substrate, fiber or window. When layered, the photochromic or photobiologic material of the top most layer first reacts and gives off a by-product which cause a reaction in the second lower layer and the change in property of the second layer can then be measured. In non-hermetically sealed sensor assembly with an air flow mechanism, the first reaction can occur by providing the first photo-chromic or photo-biologic material in the intake filter of the mechanism.

Also, other analog compound(s) may be provided on the sensing element in addition to the one or more photochromic or photobiologic material(s) to be sensed so as to differentiate detection of analog compound(s) and the actual compound(s) desired to be sensed by the photochromic or photobiologic material. In other words, the other sensing material is used in the same sensor to senses second specific analytic having receptor sites which bound molecularly to another specific analytic when present, in which the specific analytics of photochromic or photobiologic material and the analog material are molecularly similar to each other. This can avoid confusion between molecularly similar compounds, in which one could be dangerous and the other not, thereby avoiding false positives. When material for sensing different compounds are provided on the same sensing element, the compounds may be in bands on strips on the sensing element, e.g., substrate, fiber or window.

The photochromic or photobiologic material may be mixed with a metal organic ligand, where the metal is one of platinum, nickel, or metals which have unsaturated valence shells in the P, D, or F orbital's, and the ligand is of the general formula which contains at least one unsaturated carbon double bond. The sensing material may also be an impregnated organic polymer.

Both non-hermetic and hermetic sensor unit assemblies may utilize fullerenes to support their elements, such as their sensing, filter, or reacting elements. The fullerenes can be sheets, tubes, or bucky balls.

### BRIEF DESCRIPTION OF THE DRAWINGS

The foregoing and other features, and advantages of the invention will become more apparent from a reading of the following detailed description in connection with the accompanying drawings in which:

FIG. 1 illustrates the operation of a photochromic molecular switch;

FIG. 1A is a top view of a substrate of the sensor unit;

FIG. 1B is a cross-sectional view from taken through the openings of FIG. 1A;

FIG. 2 is a plan view of the substrate along the lower surface of FIG. 1B;

FIG. 3 is a plan view of the substrate along the upper surface of FIG. 1B;

FIG. 4A is a top view of the sensor carrier showing an example eight grooves for receiving sensors;

FIG. 4B is a cross-sectional view from taken through the length of FIG. 4A showing one or the channels or grooves;

FIG. 4C is another cross-sectional view from taken thorough the width of FIG. 4A showing the V cross sectional shape of the grooves;

FIG. 5 is a block diagram of the sensor unit assembly of the present invention with one of sensor element being shown having the substrate of FIGS. 1A and 1B, sensor carrier of FIGS. 4A-4C, and a mechanism enabling air flow between the carrier and the substrate;

FIG. 5A is schematic view of the sensing element of FIG. 5 for the example of an optical fiber with single coating or layer of photo photochromic or photobiologic material;

FIG. 6 is partial view of the sensor element to show the light path along the sensor element;

FIG. 7 is a block diagram showing another assembly of the reverse reaction sensor of the present invention using a hermetic sealed sensor assembly housing;

FIG. 8 is top view of a substrate similar to FIG. 7 with structures that provides for interferometric detection;

FIG. 8A is side view of sensor unit assembly similar to FIG. 7 using the substrate of FIG. 8 for an example of one of light paths of FIG. 8, in which the pair of directors to and from the window in the waveguide of FIG. 7 are provided instead by a pair of etched pits lowermost in FIG. 8;

FIGS. 9A and 9B are side and cross-sectional views, respectively, of multiple compounds upon an optical fiber in strips to provide a sensing element such as used in FIG. 5; and

FIG. 10 shows multiple compounds on an optical fiber in bands to provide a sensing element such as used in FIG. 5.

### DETAILED DESCRIPTION OF THE INVENTION

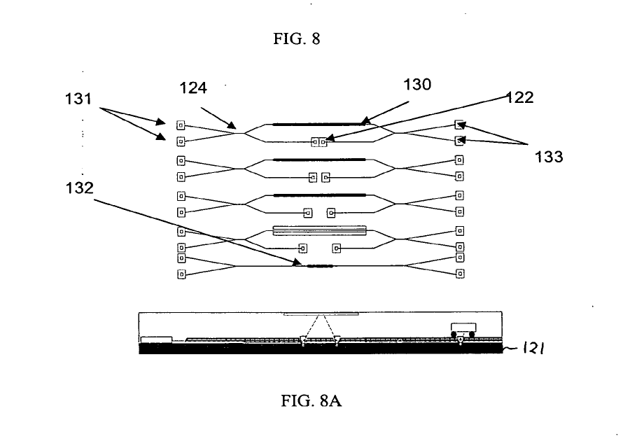

The sensor of the present invention is constructed by implementing various coatings which include substituted chromic and biologic substances which are specific to a certain compound or class of compound, or element. When irradiated with an appropriate wavelength energy these chromic or biologic materials undergo changes to their elemental structure. The change in structure enable airborne compounds of interest to become chemically bonded to the surface of the glass bead, fiber, or reflective surface providing the sensing element. When this happens, the absorption spectrum of the compound changes, and this change in can be measured through monitoring of the absorption spectrum of the compound at a given wavelength, or change in refractive index, or color. Any change that can be measured by spectrometric methods can be used as long as the reaction taking place on the surface is reversible. Many chromic reactant species have structural changes when illuminated and the change reverses when the illumination is removed or changed to another frequency. In the stimulated state the desired chemical will be bound to the surface to the material. When the reaction is reversed, the sensor material will release their bound air borne materials and these will be swept away in the stream of air which is flowing across the sensor. In some applications the environment is so sever that it would be desirable to have a sealed sensor system and have the light pass through a window and interacts with a coating or polymer which is on the surface of the window onto a coating. Such sealed sensor unit assembly will be described latter in connection with FIGS. 7 and 8.

Sensors assisted reversible reactions or photochromic selectivity have a clear advantage over mass spectrometry based analysis sensing technology of airborne compounds.

Photo-chromic molecular switches is a molecule that can be reversibly shifted between two or more stable states' the molecules may be shifted between the states in response to changes in e.g. pH, light, temperature, an electrical current, microenvironment, or the presence of a ligand. In some cases, combinations of stimuli are utilized.

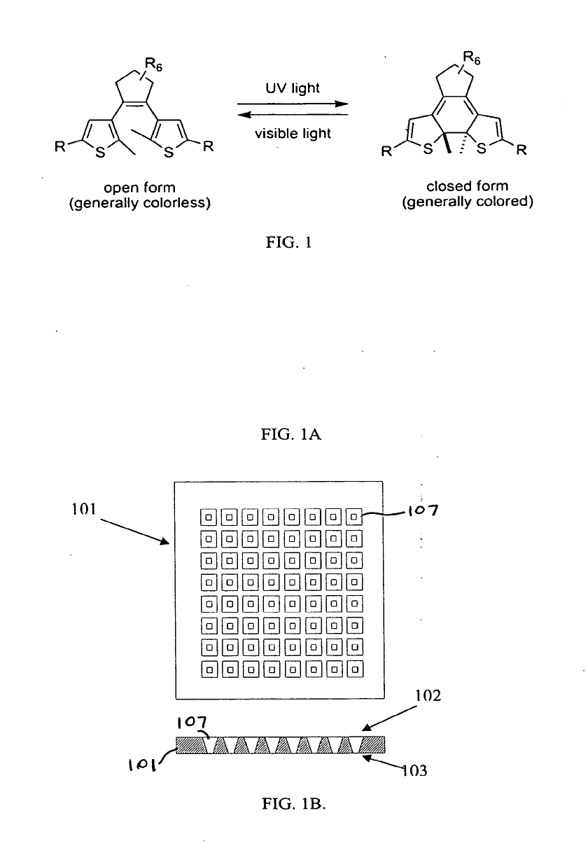

Photochromic molecular switches are a widely studied class of compounds which are able to switch between electronic configurations when irradiated by light of a specific wavelength. Each state has a specific absorption maximum which can then be quantitatively measured using UV-VIS spectroscopy. Members of this class include, but are not limited to azobenzenes, diarylethenes, dithienylethenes, fulgides, stilbenes, spiropyrans and phenoxynaphthacene quinones. An example of a photochromic molecular switch is dithienylethenes as shown in FIG. 1. This molecule is red when exposed to UV light, and when exposed to white light, is colorless.

A sensor may be constructed from dithienylethenes coated on a disk made of quartz or a sensing element, such as a quartz fiber, and placed in the sensor path trench or coated on the channel or V-shaped groove 115, or applied upon a window 126, as will be shown below. When illuminated by white light, the coating will lose color and absorption of light at wavelengths specific to dithienylethenes in the open form will ensue. However, in the presence of based and compounds such as found when sensing typical by-products given off by munitions and explosives, binding will occur across the open molecular bridge. Because of this, the dithienylethenes can be put into a state which will allow for binding by application of heat or photons of the appropriate frequency of light so that the compounds will bind on the surface. As this occurs, the absorbance which normally occurs with the open bridge state will be reduced. The reduction will be proportional to the concentration of nitrogen and nitrogen containing compounds in the air. Since the surface area of the coated lens, fiber, V-grove or window is known, the concentration is determined by application of basic analytical chemistry techniques. Normal air contains naturally occurring amounts of nitrogen and oxygen. However, nitrogen containing compounds, oxygen and in some instances chlorine concentrations measured in the presence of explosives are much higher than background. Table 1 show the relative elemental densities of hydrogen, carbon, nitrogen, chlorine, carbon monoxide, cyanogens, and oxy-chlorine normally found around explosives, narcotics, and plastics relative to background.

TABLE 1
Elemental densities and ratios of three classes of substances.

| | Density or Ratio | | | | | | | |
|---|---|---|---|---|---|---|---|---|
| | H | C | N | O | Cl | C/O | C/N | Cl/O |
| Narcotics | High | High | Low | Low | Medium | High, >3 | High | Very |
| | | | | | | | | High |
| Explosives | Low- | Med | High | Very | Medium | Low, | Low, <1 | Low to |
| | Medium | | | High | to None | <1 | | Medium |
| Plastics | Medium- | High | High to | Medium | Medium | Medium | Very | — |
| | High | | Low | | to None | | High | |

By utilizing photochromic or photo-biologic compounds, the reversible reaction sensor of the present invention can be made is sensitive to a wide variety of elements. When put in the active state, the time it takes for the activated compound to reach a maximum level of absorbance, color change, or whatever property measured is monitored. The maximum state is reached when no further increase in the measured property takes place. The sensor has a known theoretical maximum based on surface area, and concentration per unit area of the reactive species. When de-activated the sensor releases the compound of interest and return to the normal state and can be re-activated again. Monitoring saturation over time provides a quantifiable measurement of relative concentration. In low concentrations areas the sensor will take longer to saturate than in higher concentration areas. Specialized time based rate sensors are provided by substituting metal-organic ligands, metals themselves, or incorporating mixtures of biologic compounds with the chromic compounds. In the sensor construction, the concentration of reactive substances may be varied, yielding multiple sensors with various concentration levels of the same, or different reactive materials on the surface creating differential analytical systems. The relative concentration change determined enables multiple grooves 115 or windows 126 with various concentration to monitor both high and low level concentration changes. Combining several different reversible reaction chemistries into several grooves 115 of a substrate or upon windows 126, selectivity, and sensitivity to several different compounds can be sensed.

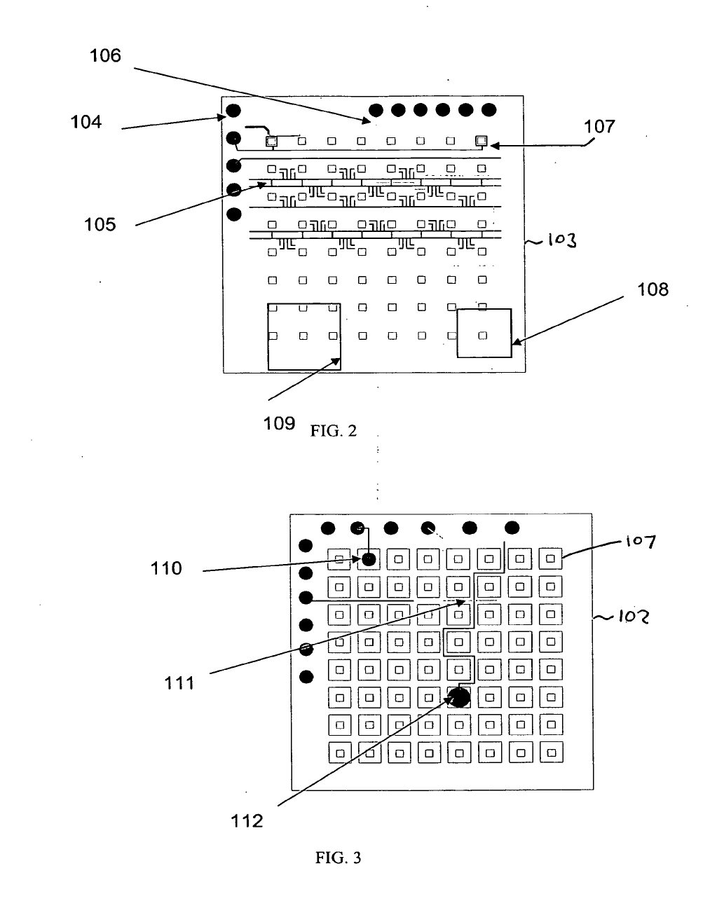

Referring to FIGS. 1A and 1B, a micro machined silicon substrate 101 is a 1, 0, 0 crystal orientation silicon substrate with a first surface or side 103 and a second surface or side 102. The first surface 103 of FIG. 1B is shown in FIG. 2, and shows an example of routings of metallization, such that parallel electrical configuration 105, that can be patterned along with additional metal layers 106 through the deposition of dielectric materials. Additionally, base metals can be deposited around the openings 107 in the silicon and solder balls 104 can be attached to facilitate electrical and structural connections, such as flip chip attached or wire bonded semiconductor devices 108 and 109. The second surface 102 of FIG. 1B is shown in FIG. 3 and shows metallization with crossovers 111, solder bumps 110 may be attached on the periphery and in the etched holes 110 to facilitate electrical contacts from the first surface 103 to the second surface 102. Ball 112 is composed of metals, or sapphire, or another element or compound which can be used as a lens, sensor element or structural alignment between layers of silicon substrates, or as an electrical contact to a sensor element. Additionally, the ball 112 can be used to launch an RF signal. The particular structure and arrangement of electrical and optical components on surfaces 102 and 103 are provided to enable operation of the sensing in the sensor, where different electrical routing 105 and 106 and solder bumps 104 on either the first or second side may be provided to enables connectivity , and connections from layer to layer of micro machined silicon.

The substrate 101 may represent a universal optical interconnect substrate with beveled or isotropic pathway micro-machined into the silicon between surface 102 and 103 going part or all of the way from the first to the second side 102. The non-etched portions of surfaces 103 and 102 are utilized for the mounting of light sources and detectors.

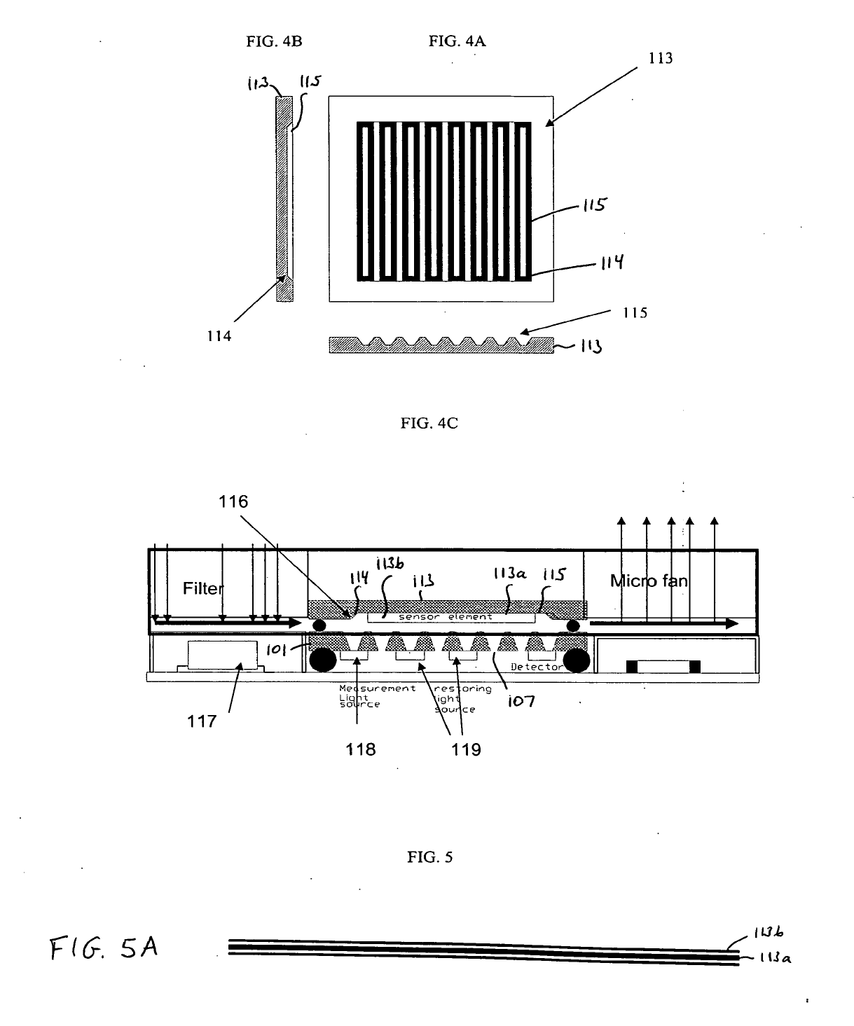

A holder or sensor carrier 113 for the smart sensor is shown in FIGS. 4A, 4B, and 4C. The sensor carrier or member 113 is constructed of 1, 0, 0 crystallographic orientation silicon and has channels or V cross-sectional shaped grooves 115 micro machined into the surface. The end surfaces 114 of the grooves can be metalized and used to reflect light down the groove to the other end surfaces of the grooves. The grooves 115 may form by micro machining with or isotropic etching.

A metallic coating is selectively deposited on either the first, second, or both sides of the substrate 101 which provide both an electrical interconnect pathway, and a path for light 114 to and from grooves 115. Additionally several metallic coatings could be selectively applied to a given surface as desired. Some of these coatings could include marker, or reactive species which bind with certain elements, or molecular species to cause changes in refractive index, absorption spectra, or color change.

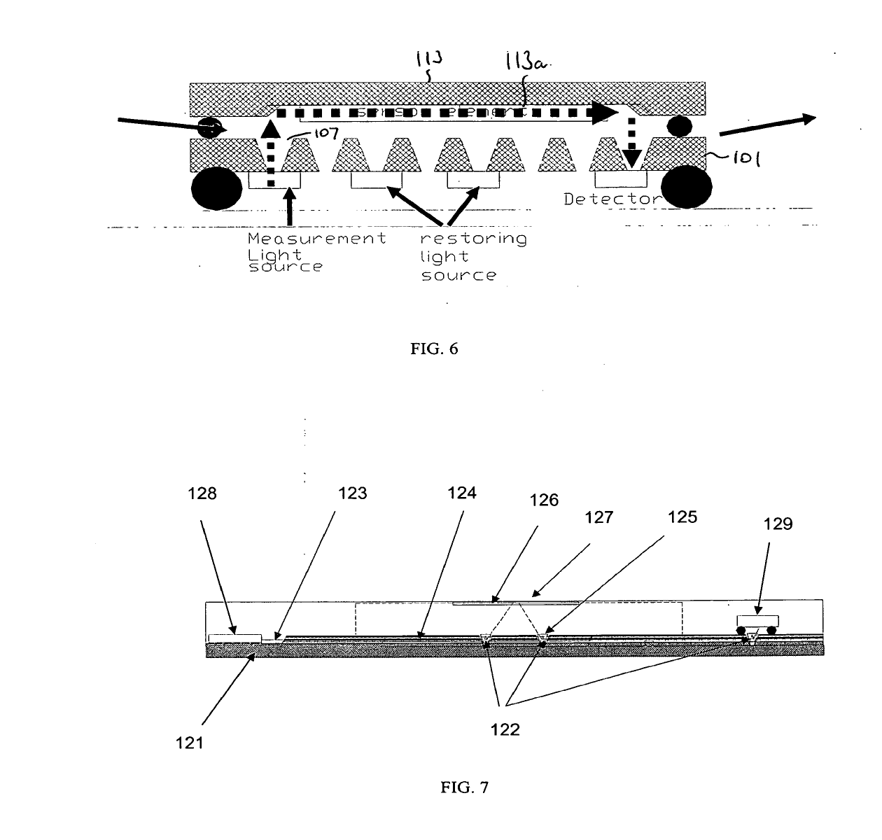

Referring to FIG. 5 there is shown the sensor unit assembly for one of the grooves 115 having sensor element 113a with a sensing material 113b on its surface, such as a polymer, of one or more photo-chromic or photo-biologic compounds for a specific analytic having receptor sites which bound molecularly to a specific analytic when present. The sensing material may be a layer, coating, multiple layers, and having other configurations as shown in FIGS. 10A-C, and 11. The carrier 113 facing surface 102 of substrate 101 and has a space or region therebetween for air flow. Light that is created from LED's, lasers, or other sources 118 is mounted on surface 103 of substrate 101 and passes through the etched silicon openings or holes in 107 and strikes a mirror or reflective surface 116 formed in end surface 114 of groove 115. The light which strikes etched and metalized surface 116 is re-directs into and along the groove 115 and thus coupled into sensing element 113a. The sensing element 113a light may a fiber, glass bead, or other material which allows light to pass through or along the groove 115. The sensing element has on its surface the photochromic or photobiologic material(s) described earlier to provide a reverse reaction sensor. Alternatively, the groove 115 itself can act as the sensing element provided it is coated with material(s) which create a reverse reaction sensor. The numbers of fibers or sensors that can be realized per unit area are only limited by lithographic methodologies for any given construction. FIG. 6 illustrates by dashed arrows the path of light from source to detector along sensor element 113a in groove 115.

In the example of FIG. 5, the sensor element 113a is a bare quartz fiber with a photochromic or photo-biologic coating 113b as shown in FIG. 5A. Source 118 is a Light Emitting Diode (Led), a Super-luminescent Light Emitting Diode, or (SLED) semiconductor laser or other device emits light of wavelengths in from the UV spectrum to radio waves. Item 119 delineates another source of light, RF or it could be a series of detectors. The sensor assembly of FIG. 5 may be in a housing having supports for substrates 101 and 113, as well as for other components as shown. For example, such support may be provided by tubes on either side of the air flow region as shown, or other fullerenes structures may be used.

If the sensor is designed is to be used in harsh environments, the lower micro machined silicon substrate 101 can be complete sealed by a quartz cover slip. Alternately, the micro machined holes 107 can be filled with epoxies which allow light or other radiation for operating the sensor to pass through. A micro fan and filter provide for air flow 120 across sensing element 113a, but other mechanisms providing air flow may be used. The sensor unit shown in FIG. 6 may be in a housing that is not hermetically sealed to enable air flow.

Another sensor unit assembly may be used when surface measurements are warranted or the environment is so harsh that the flow sensor cannot be used. This hermetic sealed sensor assembly is shown in FIG. 7. A silicon substrate has etched V-grooves 121 which has a light source(s) 128 and a detector(s) 129 mounted on the surface. Other components electrical or optical can be mounted as well. The substrate may be a single piece of micro machined silicon. A beam of light of an appropriate wavelength(s) 123 is directed into a deposited waveguide(s) 124 and the light is guided to a V-groove where it encounters a specially made and mass produced quartz optical director device 122. The light is directed from the waveguide onto the surface of the optical window 126 where it encounters a polymer 127 which has been prepared to be sensitive to the materials in question. The light beam 126 interacts with the polymer 127 and absorption of light occurs. The optical window is made in such a way as to return the beam back to a precise position on the substrate and this strikes another optical director device (ODD) 122 and the light is directed back into the waveguide where it travels along until it strikes another ODD 122 and is directed into a detector where measurements are made as to the intensity or spectral composition.

Conventional devices utilize fiber and ball lenses to align the output of an LED diode to the embedded waveguide fabricated on silicon. In the assembly of FIG. 7, no fibers are needed to align the laser or LED to the waveguide 121. Moreover, passively aligned precision ground beam diverter enable the deep and shallow angle measurements required in biological tissue sampling techniques. This enables transmissive, absorptive, differential, and subtractive measurement and resides on one substrate. A wider variety of measurements can be made which ensures accuracy. Some pathways flow straight across the substrate, and are the baseline reference standard, or can be used to interfere with the light which passes through the sensor portion. Additionally some pathways could direct the light to the second side, and all pathways can have various, known path lengths by design. The pathways can be controlled to pass or block the light entering the network either by an on-substrate controlling methodology or by controllers integrated by flip chip, or wire bonding. However any other interconnection methodology may be used. The methods that can be used to switch the pathways on or off vary widely depending on the materials used in the construction of the interconnection device.

Referring to FIG. 8 is shown a view of the substrate 121 similar to FIG. 7 but with waveguides which provides for interferometric detection of changes in the sensing material on the window. Deposited waveguides 124 form a dual Y-branch which acts as a 3 dB splitter/coupler. Exiting the Y-branch is another Y-branch which allows approximately ½ of the light to pass through either pathway to the other side where it is combined again for readout at the detector 133. Detectors and sources can be combined such that the sources are at either end 131 and/or 133, or a combination of detector and source are at both ends. Having the ability to source and detect at each end provides capability that has not existed in other embodiments. Each pair of waveguides has a pair of etched pits 122 and a pass-through region 130. In the pass-through region there are metals deposited under the waveguide 130, these metals can act as a resistive heater, or, with proper materials deposition techniques can shift the light using a magnetic field. The heating of the waveguide or the use of a magnetic layer will phase shift the light passing through the waveguide above. It is this shifting of the light that eliminates the need for a moveable mirror to cause interference patterns to exist when the light is re-combined which greatly simplifies the requirements in the measurement system. FIG. 8A is side view of sensor unit assembly similar to FIG. 7 showing for an example of one of light paths using the pair of etched pits 122 lowermost in FIG. 8.

Optical element 132 enables the reference beam and sample beam to the same. In the other structures, a reference beam passes through a separate channel and is re-combined on the opposite side from launch. However, in element 132 the light passes though a ½ silver free-space optical splitter/combiner which enables the reference beam and sample beam to be one in the same. This reduces the overall number of component in the system and can reduce the overall size of the system by 50% or more.

The entire silicon interconnection of the present invention can be fabricated in batch, using current practices utilized in high volume semiconductor manufacturing facilities, and silicon optical bench manufacturing facilities. The accuracy of the V-groove center to the center of the fabricated waveguide can be extremely high (one micron or less), and completely eliminate the need for active alignment of fibers, free space optical splitters/combiners, or optical pathway fiber sensor devices. All construction pieces can be passively aligned which allows for high throughput manufacturing.

Other elements may be provided in the sensor units shown in FIGS. 5 and 7 to provide power to the components of the sensor, and control lines to operate radiation sources and detectors, as well as to disable or change such operation of sources to reverse the reaction on the sensing element 113a or widow 126 when needed to self clean sensors of the units. The detectors may be a typical optical detecting device, such as a photodetector, CCD, RF receiver, or the like, which can detect the desired property or characteristic which determines the presence, absence, or any change when molecular binding to the compound(s) or analytic(s) of interest occurs in the photochromic or photobiologic material(s) along the sensing element 113a. Electrical signals from detectors in the unit may be provided to electronics (e.g., programmed processor) to measure the presence or level of the compound(s) detected by the sensor, and provide output (LCD) to users with the results, as typical of chemical compound sensing devices.

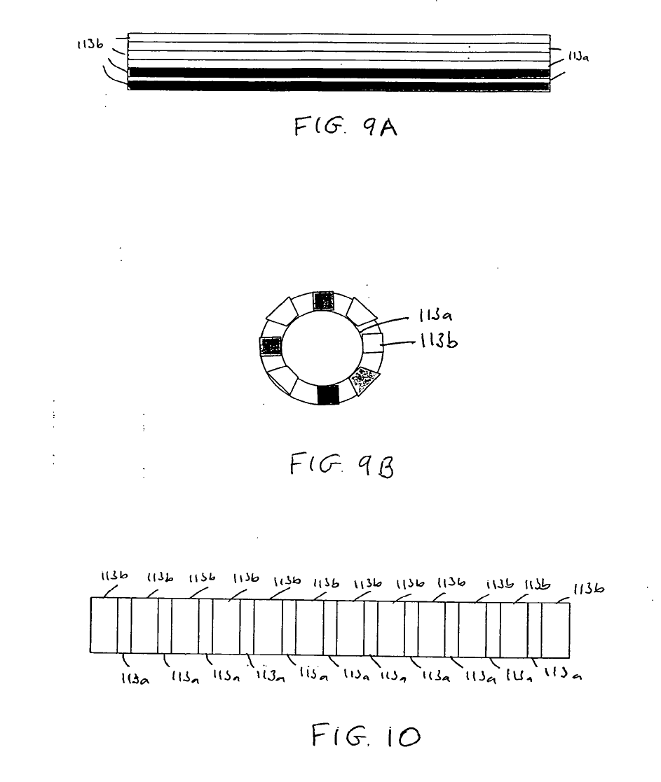

Multiple compounds may be provided on the same sensing element, e.g., substrate, fiber, channel (groove), or window. One or more of these photochromic or photobiologic material(s), other may be molecularly analog compounds as described earlier to avoid false positives. FIGS. 9A and 9B show multiple compounds on the example of fiber sensing element, where different stripes can be the same or different compounds. FIG. 10 shows multiple compounds is bands along a fiber. However, other orientation of the compounds may be used each are aligned with illumination and detecting mechanisms in the sensing unit of FIG. 5 to detect changes in properties indicating the presence and/or level the particular compounds.

From the foregoing description, it will be apparent that a reversible reaction sensor device and sensor unit assemblies is provided. Variations and modifications of the herein described sensor, assemblies, and methods for implementing will undoubtedly suggest themselves, to those skilled in the art. Accordingly the foregoing description should be taken as illustrative and not in a limiting sense.

1-23. (canceled)

24. A reversible reaction sensor device comprising:
a material for a specific analytic having receptor sites which bound molecularly to said specific analytic when present;
a source for radiation;
a waveguide having said material, in which said radiation passes along said waveguide; and
a detector for sensing a change in an optical characteristic of said material when exposed said radiation passed along said waveguide, in which said change is caused by said receptor sites being bound to said specific analytic.

25. The device according to claim 24 wherein said detector senses said specific analytic in accordance with a response time to saturation of the material when exposed to the radiation, and said binding of said specific analytic to said receptor sites is reversible.

26. The device according to claim 24 wherein said waveguide is provided by a channel or groove formed along a surface of a silicon substrate, said channel or groove has a reflective surface to reflect the radiation provided by said source down along a length of said channel or groove toward said detector.

27. The device according to claim 26 wherein said channel or groove is V-shaped.

28. The device according to claim 26 wherein said channel or groove has two ends each with an angled surface, wherein one of said angled surface at one end directs said radiation from said source toward the other of said ends, and said angled surface of said other of said ends directs radiation from said channel of groove to said detector.

29. The device according to claim 26 wherein said material is one of present upon said reflective surface of said channel or groove, or provided by an optical element along said channel or groove.

30. The device according to claim 24 wherein said change in said optical characteristic is one of the absorption spectrum of the compound on the surface, refractive index, or color.

31. The device according to claim 24 wherein said radiation is light.

32. The device according to claim 24 wherein said radiation represents first radiation, and said sensor further comprises a restoring source operable to provide second radiation to said material that releases said specific analytic when bound to said receptor sites for cleaning said sites of said specific analytic.

33. The device according to claim 24 having a plurality of ones of said waveguide for sensing a specific analytic in which each has a different one of said detector and said source.

34. The device according to claim 24 wherein said detector operates to measure presence or level of said specific analytic.

35. The device according to claim 24 wherein said specific analytic represents one or more airborne compounds.

36. The device according to claim 24 wherein material is self cleaning by one or more of radiation of a specific wavelength, specific frequency, absence of said radiation, which causes the receptor sites to close and the bound specific analytic are released.

37. The device according to claim 24 wherein said material is a photo-chromic or photo-biologic compound.

38. The device according to claim 24 wherein said material is mixed with a metal organic ligand, or is of an impregnated organic polymer.

39. The device according to claim 24 wherein said material represents a plurality of photo-chromic or photo-biologic materials in which one of said photo-chromic or photo-biologic materials causes a first reaction to occur which releases a compound causing a second reaction in another of said photo-chromic or photo-biologic materials, and said second reaction effects said optical characteristic.

40. The device according to claim 39 further comprising a fan providing an air flow along said waveguide via an intake filter, and said intake filter has said one of said photo-chromic or photo-biologic materials for causing said first reaction, and said waveguide has said another of said photo-chromic or photo-biologic materials for causing said second reaction.

41. A reversible reaction sensor device comprising:
a material for a specific analytic having receptor sites which bound molecularly to said specific analytic when present;
a source for first radiation;
a waveguide having said material, in which said first radiation passes along said waveguide;
a detector for sensing a change in an optical characteristic of said material when exposed to said first radiation passed along said waveguide, in which said change is caused by said receptor sites being bound to said specific analytic;
a housing having said source, said waveguide, said detector, and means for providing an air flow along said material to expose said material to said specific analytic when present; and
a restoring source operable to provide second radiation to said material that releases said specific analytic when bound to said receptor sites.

42. The device according to claim 41 wherein said waveguide is provided by a channel or groove having a reflective surface to reflect said first radiation provided by said source down along a length of said channel or groove toward said detector, and said material is one of present upon said reflective surface, or along an optical element in said channel or groove.

43. The device according to claim 41 wherein said material represents a plurality of photo-chromic or photo-biologic materials in which one of said photo-chromic or photo-biologic materials causes a first reaction to occur which releases a compound causing a second reaction in another of said photo-chromic or photo-biologic materials and said second reaction effects said optical characteristic, and said housing further comprises a filter for filtering said air flow received by said waveguide, said filter has said one of said photo-chromic or photo-biologic materials for causing said first reaction, and said waveguide has said another of said photo-chromic or photo-biologic materials for causing said second reaction.
